(19) United States
(12) Patent Application Publication
(10) Pub. No.: US 2022/0280165 A1
(43) Pub. Date: Sep. 8, 2022

(54) MEDICAL SET FOR TREATING ANEURYSMS, PRODUCTION PROCESS, AND MEDICAL SYSTEM FOR TREATING ANEURYSMS

(71) Applicant: Acandis GmbH, Pforzheim (DE)

(72) Inventors: Michael Büchert, Pforzheim (DE); Eugen Schönberger, Pforzheim (DE); Giorgio Cattaneo, Karlsruhe (DE)

(21) Appl. No.: 17/632,687

(22) PCT Filed: Jul. 21, 2020
(86) PCT No.: PCT/EP2020/070500

§ 371 Date: Feb. 3, 2022

(30) Foreign Application Priority Data
Aug. 9, 2019 (DE) 102019121554.4

(51) Int. Cl. A61B 17/12; A61F 2/90

(52) U.S. Cl. CPC A61B 17/12118; A61B 17/12177; A61F 2/90; A61B 17/12145; A61F 2002/823

(57) ABSTRACT

The invention relates to a medical set for treating aneurysms, including a main catheter and a covering device which can be moved through the main catheter to a treatment site and is intended to cover an aneurysm. The covering device is connected or can be connected to a transporting wire and includes a self-expandable grid structure made up of webs that are connected to one another in one piece and delimit inner cells and outer cells. The outer cells form at one longitudinal end of the grid structure a closed outer-cell ring, which is connected only on one side to inner cells. The grid structure is provided with a covering made of a fabric which has pores of irregular sizes, wherein at least one inner cell of the grid structure is at least partially, in particular mostly, free from the covering. A production process and a medical system are also provided.

The invention relates to a medical set for the treatment of aneurysms in accordance with the preamble of patent claim 1. Furthermore, the invention relates to a production method as well as to a medical system.

WO 2014/177634 A1 describes a highly flexible stent which has a compressible and expandable mesh structure, wherein the mesh structure is formed in one piece. The mesh structure comprises closed cells which are each delimited by four mesh elements. The mesh structure has at least one cell ring which comprises between three and six cells.

Furthermore, to the Applicant's knowledge, stents with mesh structures are known which are formed from a single wire. The wire is braided with itself in order to form a tubular network. At the axial ends of the tubular network, the wire is curved round so that loops which act atraumatically are formed. The axial ends may flare outwards in a funnel shape.

The known medical device is particularly suitable for the treatment of aneurysms in small cerebral blood vessels. Blood vessels of this type have a very small cross sectional diameter and are often highly tortuous. For this reason, the known stent is highly flexible in configuration, so that on the one hand it can be compressed to a very small cross sectional diameter and on the other hand it has a high bending flexibility, which enables it to be delivered to small cerebral blood vessels.

For the treatment of aneurysms in cerebral blood vessels, it is advantageous to use stents which bridge an aneurysm and screen it from the flow of blood inside the blood vessel. To enable this, providing stents with a covering is known; it occludes the cells of the stent and thus prevents the flow of blood into an aneurysm as well as perfusion from side branches of the blood vessel.

A further supplementary or alternative method for the treatment of aneurysms is the implantation of what are known as coils into the aneurysm which, once there, lead to the production of a blood clot. The resulting thrombus then prevents blood from circulating in the aneurysm and thus removes the risk of a rupture with accompanying haemorrhage.

In particular in the case of broad-neck aneurysms, however, the coils have a tendency to displace during implantation into the blood vessel and thus cause the main vessel lumen to become occluded. In the “Balloon Assisted Coiling” technique, catheters are positioned in the vessel with “Compliance Balloons”, in particular at the level of the neck of the aneurysm. The balloon, which is filled with contrast agent, then occludes the neck of the aneurysm during placement of the coil and forces the coil into a compact arrangement inside the aneurysm sac. Because coils are plastically deformable, they then remain in their shape. In addition, they do not come out of the aneurysm when the balloon is removed. However, a problem arises this regard, in particular because of the fact that the balloon occludes the vessel. In a lengthy procedure (in the case of large aneurysms, a plurality of coils are positioned, and the procedure can last several minutes), the flow of blood is completely cut off during that time. Even though collateral vessels supply neighbouring tissue, the risk of underperfusion still arises.

Furthermore, during the procedure a catheter through which the coils are fed is “jailed”, i.e. wedged at the side of the balloon. In the event that the coil catheter has to be changed (for example in the case of damage) when the procedure has not yet been completed, the balloon has to be deflated (emptied) in order to be able to withdraw the catheter. In this phase, coils which are not yet fully compacted in the vessel could become displaced. This could result in occlusion of the vessel.

In the light of the foregoing, the objective of the invention is to provide a medical set for the treatment of aneurysms with the aid of which the risk of occlusion of side branches of a blood vessel is at least reduced. A further objective of the invention is to provide a method for the production of a medical set, as well as a medical system.

In accordance with the invention, this objective is achieved in respect of the medical set by the subject matter of patent claim 1, in respect of the method for the production of a medical set by the subject matter of patent claim 11 and in respect of the medical system by the subject matter of patent claim 12.

The subject matter of the dependent claims pertains to preferred embodiments, further embodiments and variations.

Specifically, the objective is achieved by means of a medical set for the treatment of aneurysms, having a main catheter as well as having a covering device which can move through the main catheter to a treatment site. The term “treatment site” as used here should be understood to mean a position along a vessel at which the aneurysm is formed. In this respect, the covering device serves to cover the aneurysm. Furthermore, the covering may be a temporary cover for the aneurysm, for example only for the length of time during which an embolization means is placed inside the aneurysm. Particularly preferably, the cover is long-lasting, i.e. permanent, for example in the form of permanent implantation of the covering device into the vessel, in particular the form of a stent or a flow diverter.

The covering device may be mechanically connected to the transport wire in a fail-safe manner, i.e. non-detachable. In the case in which the covering device is a permanent. implant, for example forming a stent or a flow diverter, the connection to the transport wire may also be mechanically detachable. Furthermore, the covering device has a self-expandable mesh structure produced from webs. The term “self-expandable” as used here should be understood to mean that the mesh structure can pass from a compressed state into an expanded state without the intervention of external forces. In this regard, the mesh structure preferably includes a shape memory material or is formed from a material of this type.

The webs of the mesh structure are unitarily connected with each other and delimit inner cells as well as edge cells, wherein the edge cells at one longitudinal end of the mesh structure, for example at a distal and/or proximal longitudinal end of the mesh structure, form a closed edge cell ring. The edge cell ring in this respect is connected to the inner cells on only one side. The term. “edge cell ring” as used here should be understood to mean a ring of cells which are adjacent in the circumferential direction, in particular edge cells.

Furthermore, the mesh structure is provided with a covering produced from a fabric. In this regard, the fabric has irregularly sized pores. At least one inner cell or the mesh structure in this regard is partially and in particular mainly covering-free, i.e. it is not provided with the covering or with a portion of the covering. The term “mainly covering-free” as used here should be understood to mean that a continuous surface, in particular of more than half of the total surface area of the mesh structure, is covering-free.

The advantage in this case is that on the one hand, a flow of blood in the vessel, for example during placement of a coil, is guaranteed because of the longitudinal axial perfusibility of the mesh structure by blood. On the other hand, a reliable and especially location-specific covering of the aneurysm is ensured without side branches being covered by the covering, however. A further advantage lies is the reduction in the total surface area of the covering.

Because of the longitudinal perfusible mesh structure, then a flow of blood, in particular in the longitudinal direction through the blood vessel, is barely hindered, but a flow of the blood into a branching aneurysm is prohibited by the covering or at least, ingress of a flow into the aneurysm is reduced. In particular in the case of a permanent implantation of the covering device, this longitudinal perfusibility of the covering device has been shown to be particularly suitable. A possible and unwanted occlusion of the vessel, as can occur, for example, with the aforementioned prior art using a balloon, is therefore at least reduced and preferably excluded. By means of the at least one covering-free inner cell, furthermore, a supply to the underlying vessel tissue is also enabled.

A configuration of the mesh structure of this type also enables the medical set to be used as a stent or flow diverter, which barely hinders the flow of blood in the longitudinal direction and also, for example, to side branches through the blood vessel, however ingress of the blood into a branching aneurysm is prevented by the covering, or at least the ingress is reduced. A possible and unwanted occlusion of the vessel as can occur, for example, in the aforementioned prior art using a balloon, is therefore at least reduced and preferably excluded. Furthermore, for example, side branches next to or near the aneurysm are still perfusible with blood.

Particularly in the case of fusiform aneurysms, i.e. aneurysms which extend over the entire circumference of a blood vessel, the use of a deliberately fine-pored structure is advantageous for the colonization of endothelial cells. In this manner, a reconstruction of the missing vessel wall can be obtained. Specifically, the structure provided with a specific pore size which is formed by the electrospun fabric, forms a scaffold for colonization by endothelial cells which can then form a new, closed vessel wall. The term “fusiform aneurysms” as used herein should be understood to mean aneurysms which extend over at least 50%, in particular over at least 75% of the total circumference or over the entire circumference of a blood vessel.

In a preferred embodiment, the covering is formed from an electrospun fabric.

In an electrospun fabric, pores are usually irregular in shape. However, the production method does not permit pores to be produced with a specific arrangement or shape. In fact, the pore size can only be adjusted with the aid of the process parameters to the extent that it is ensured that at least a portion of the pores have a certain minimum size.

As an example, the electrospinning process may be carried out directly on the mesh structure so that a bond with the mesh structure is produced at the same time as the covering is formed. The covering may be mechanically interlocked with the mesh structure. As an example, the covering may be bonded to the mesh structure by means of an adhesive bond. The adhesive bond may be produced by means of a bonding agent. The bonding agent may, for example, comprise polyurethane or consist of polyurethane.

Furthermore, the covering produced from an electrospun fabric is extremely thin and flexible, which adds to the flexibility of the mesh structure. In particular, the covering barely inhibits compression of the mesh structure, which is in contrast to prior art coverings which are produced from textile materials. Overall, then, the entire covering device can be compressed to a considerably smaller cross sectional diameter, and thus can be fed via small catheters into particularly small blood vessels. This is of particular relevance for the treatment of aneurysms in cerebral blood vessels; the invention is particularly suitable for this purpose.

Preferably, the covering is formed by a synthetic material, in particular by a polymer and preferably by polyurethane. Materials of this type are particularly light and can be readily produced in fine filaments by means of an electrospinning process. On the one hand, the synthetic material can therefore be used to produce a particularly thin and fine-pored covering. On the other hand, the synthetic material already has a high intrinsic flexibility, so that a high compressibility of the medical set is obtained. Alternatively, the covering may also be produced from polyethylene or fluoropolymers.

Thus, treatments are also possible using the medical set in accordance with the invention in blood vessels which could not be accessed with medical devices of the prior art which nave a mesh structure and a covering. Because of the high compressibility of the device in accordance with the invention, very low delivery forces are generated when delivering via a catheter. In addition, the material of the covering may contribute to a reduction in the delivery forces. In particular, the delivery forces in the case of the device with the covering may be the same as or smaller than delivering the mesh structure alone.

Furthermore, by means of the covering device which is appropriately disposed at the treatment site, i.e. at the level of the aneurysm, escape of the coil from the aneurysm during and after positioning of the coil can be prevented, so that the risk of occlusion of the vessel caused by the coils may also be at least greatly reduced, and preferably excluded. Especially when the covering device is configured as a flow diverter, i.e. as a permanent implant, insertion of the coils into the aneurysm can be dispensed with, because the covering preferably permanently covers the aneurysm, in particular fluid dynamically.

A further area of application for a partially covered device is fistulae, dissections and other malformations, for example arteriovenous malformations, in which the flow has to be slowed down or stopped at specific sites, or the vessel wall reconstruction has to be aided by means of a fine-meshed structure in order to promote cell proliferation. In the open regions or in the open cells, an uninterrupted flow of blood should be guaranteed, so that side branches are perfused normally. In addition, foreign materials are reduced to those most necessary, in order to improve biocompatibility or to reduce any thrombogenicity.

In order to guarantee a sufficient flexibility for the covering, it is preferably formed from irregular filaments arranged in the form of a network which have a filament thickness of between. 0.1 μm and 3 μm, in particular between 0.2 μm and 2 μm, in particular between 0.5 μm and 1.5 μm, in particular between 0.8 μm and 1.2 μm.

Particularly preferably, the medical set is configured in the manner of a stent for the treatment of aneurysms in arterial blood vessels, in particular neurovascular blood vessels. Preferably, the blood vessels may have a cross sectional diameter of between 1.5 mm and 5 mm, in particular between 2 mm and 3 mm. It is also possible to treat blood vessels with a cross sectional diameter of 4 mm to 8 mm. Carotid arteries, for example, have cross sectional diameters of this type.

In a further embodiment, all of the webs of the inner cells are respectively associated with a further inner cell or edge cell. Furthermore, the edge cells respectively have two webs which are not associated with other inner cells or edge cells. In this manner, the webs in this embodiment form both a boundary for an inner cell as well as a boundary for their adjacent edge cells.

In general, the mesh structure may be configured as a single-piece mesh structure. The braided wire of the mesh structure may consist of a single wire which curves around at the longitudinal ends of the network structure and returns. The wire may be braided with itself in order to form the network structure. The network structure may consist of a plurality of wires which are braided together. The plurality of wires may be curved and returned at one axial longitudinal end while the opposite axial longitudinal end may have wire ends which are open. It is also possible for the interwoven wires to have open wire ends at both axial longitudinal ends.

The wire may have a radiopaque core material and a sheath material produced from a shape memory alloy. In particular, the volume ratio between the core material, preferably platinum, and the volume of the whole of the composite wire is between 20% and 40%, in particular between 25% and 35%. While a braided mesh structure is characterized by a particularly high flexibility, in particular bending flexibility, a one-piece mesh structure has comparatively thin walls, so that the mesh structure influences the blood flow inside a blood vessel to a lesser extent. The mesh structure may also be laser cut in configuration. Furthermore, the mesh structure may be configured as a closed structure for withdrawal into a catheter, advantageously with between 6 and 12 cells along the circumference of the mesh structure. The cells may have an asymmetrical shape, i.e. the cells are substantially diamond-shaped, i.e. kite-shaped, but can differ from this shape by using varying web widths and lengths. However, the diamond or kite shape will still be substantially recognisable, even with these differences.

The mesh structure preferably has a cross sectional diameter of between 2.5 mm and 8 mm, in particular between 4.5 mm and 6 mm.

In accordance with a preferred variational embodiment, the covering ends at the webs of the covering-free inner cells in a manner such that the covering does not protrude into the at least one inner cell. In other words, the covering ends substantially flush with the webs, so that an edge of the covering preferably follows the contour of the webs.

By this means, on the one hand the freedom of the edge cells from a covering is guaranteed and at the same time, by placing the edge of the covering on the web, reliable securing to the mesh structure is guaranteed, for example by means of the aforementioned adhesive bond.

In accordance with an alternative variational embodiment, the covering at least partially overlaps the webs of the covering-free inner cell in a manner such that part of the covering protrudes into the at least one covering-free inner cell. In other words, in accordance with this alternative embodiment, the covering does not follow the contour of the webs exactly, but rather, it protrudes over at least 20%, in particular over at least 30%, in particular over at least 40%, in particular over at least 50% of the area of the at least one inner cell.

In both variational embodiments, however, the covering remains stable. Preferably, the covering is resistant or stable over a long period, in a manner such that the mass of the covering upon contact with blood or a physiological replacement fluid, in particular with a sodium chloride solution or Ringer's lactate solution at a perfusion rate of 100 mL to 400 mL per minute in particular, over a time period of at least four hours, in particular at least 30 days, is reduced by at most 5 %, in particular by at most 3%, in particular by at most 1%. This ensures that the action of the coating is consistent over a sufficiently long period of time.

Particularly preferably, the covering is resistant or stable over a long period, in a manner such that the mass of the covering upon contact with blood or a physiological replacement fluid, in particular with a sodium chloride solution or Ringer's lactate solution, remains completely intact over a time period of at least four hours, in particular at least 30 days. A period of time such as this, for example, enables the medical device to be coated with an endothelial layer, so that a thrombus formation is prevented in a natural manner. The antithrombogenic coating bridges the time period to natural healing or encapsulation of the medical device with a neointimal layer, in particular produced from endothelial cells, which are formed around the structural elements of the mesh.

The use of a physiological replacement fluid to test the resistance of the covering enables an objective comparison to be made. Furthermore, by using the replacement fluid, which resembles human blood, this means that objective empirical data can be determined therefrom, allowing conclusions to be drawn regarding the behaviour of the covering when in the implanted state when the covering is exposed to a flow of human blood. Thus, the replacement fluids used are preferably a 0.9 percent sodium chloride solution or Ringer's lactate solution. Replacement fluids of this type are isotonic and are suitable for use as indicators for the behaviour of the covering in the implanted state.

In accordance with one embodiment, a plurality of inner cells which are immediately adjacent in the circumferential direction of the mesh structure, in particular all inner cells of an inner cell ring, are covering-free. This ensures that adjacent side vessels and/or cells along the vessel continue to be supplied with blood, and therefore with nutrients, while the aneurysm is reliably covered by the covering. Furthermore, this means that an individual covering effect/function of the mesh structures can be provided in respect of a pattern of aneurysms disposed along the vessel so that, for example, the inner cells are provided with the covering only at the treatment sites at which an aneurysm occurs and the remaining inner cells of the inner cell ring are covering-free.

Alternatively or in a supplementary manner, a plurality of inner cells which are immediately adjacent in the longitudinal direction of the mesh structure covering-free. In this manner, the aforementioned possibility of the individual covering effect/function of the covering device is advantageously optimized.

In accordance with one embodiment, the covering extends only partially, in particular by at most 50%, in particular by at most 40%, in particular by at most 30%, in particular by at most 20% over the circumference of the mesh structure. Preferably, the covering therefore extends over the treatment site only, i.e., for example, over an opening of the aneurysm. This further embodiment means that the aneurysm is completely fluid dynamically uncoupled when using the mesh structure as a flow diverter. On the other hand, this means that in particular, cells and/or side vessels which are at the level of the aneurysm can still be supplied with blood and therefore with nutrients because of the missing covering.

In one embodiment, the covering has at least 10 pores with a size of at least 15 μm² over an area of 100000 μm². During production of the covering, this minimum size of the pores is in particular set by the duration of the electrospinning process. This combination of a specific minimum number of pores and a minimum size for these pores has in practice been shown to be particularly necessary for a sufficient perfusion or the covering by blood, for identical coverage.

In an ancillary aspect, the invention concerns a method for the production of a medical set, wherein preferably, this is the medical set described hereinabove. In this regard, the method comprises the following steps.

Firstly, the mesh structure is produced. Next, the covering is applied to all inner cells, in particular by means of an electrospinning process.

After this, at least one inner cell cut free by means of a cutting tool, wherein the cutting tool is guided along the webs of the inner cell to be cut free. The inner cell is then completely covering-free.

The cutting tool in this regard may be a mechanical cutting tool, for example a scalpel. Preferably, the cutting tool is a laser cutting tool, so that the covering is removed along the webs by means of a laser beam, so that the inner cell is cut free. Alternatively, the cutting device may also be a device which frees the at least one inner cell of the covering by means of etching.

Furthermore, in a supplemental manner, the edges of the covering may be post-processed at the covering-free inner cells. This may preferably be carried out thermally, for example by means of a soldering iron, in which in a first step, at a first temperature, rough machining of the edges is carried out and then, at a temperature which is lower with respect to the first temperature, the edges are finely machined.

In a further aspect, the invention concerns a medical system with a medical set, wherein in this case, it is in particular the medical set already described above, so that the medical set has a main catheter and a covering device which can be moved through the main catheter to a treatment site in order to cover an aneurysm.

In this regard, the covering device is connected to or can be connected to a transport wire. Furthermore, the covering device has a self-expandable mesh structure which comprises webs which are connected together into one piece and delimit inner cells as well as edge cells. At one longitudinal end of the mesh structure, the edge cells form a closed edge cell ring along the circumference of the mesh structure. The edge cell ring is connected to inner cells on only one side, i.e. on the side which is orientated in the direction of the inner cells. Furthermore, the medical system has at least one embolization means for placement in the aneurysm. Preferably, the embolization means is formed by a plastically deformable wire, in particular by a coil or a liquid. Embodiments of the embolization means of this type have been shown to be particularly suitable as regards treating aneurysms.

In the case in which the covering device is detachably connected to the transport wire, i.e. reversibly connected thereto, then particularly preferably, the covering device acts as a flow diverter for permanent implantation at the treatment site inside the blood vessel. The reversible disposition of the covering device on the transport wire is accomplished, for example, by means of a releasable mechanical lock. Furthermore, the covering device configured as a flow diverter in this embodiment has open longitudinal ends in the implanted and expanded state, and is therefore configured so as to be open at both ends. The covering device is therefore substantially cylindrical in shape, i.e. tubular, so that a flow of blood through the blood vessel is still possible. Thus, placement of the embolization means—if necessary—is possible, both in the context of implantation of the covering device as well as a later time which is subsequent to implantation of the cover ting device.

In an embodiment which is preferred, the medical system furthermore has an additional catheter for delivering the embolization means into the aneurysm, wherein the additional catheter is relatively movable with respect to the main catheter, independently of and/or in opposition to the main catheter.

The advantages and preferred embodiments of the medical set apply mutatis mutandis to the method as well as to the medical set, and vice versa. All of the dimensional information pertaining to the medical set and pertaining to the medical system is applicable to the expanded state of the mesh structure, unless otherwise indicated.

Exemplary embodiments of the invention will now be described with the aid of the accompanying diagrammatic drawings. In the highly simplified drawings:

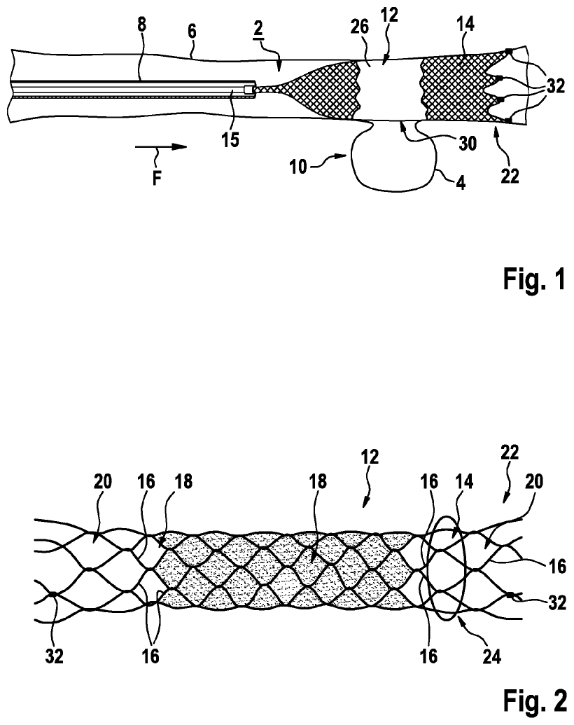

FIG. 1 shows a side view of an embodiment of the medical set in accordance with the invention,

FIG. 2 shows a close-up of a preferred embodiment of a covering device of a medical set in accordance with the invention in a preferred embodiment,

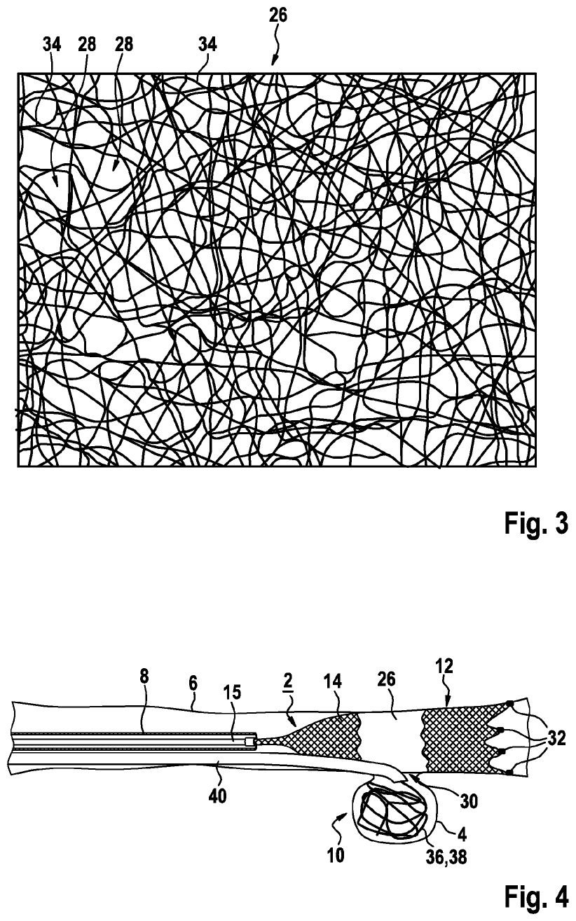

FIG. 3 shows a scanning electron microscope image of a covering of a covering device of a medical set in accordance with the invention in a preferred embodiment,

FIG. 4 shows a side view of an embodiment of the medical system in accordance with the invention in the case of temporary implantation of the covering device at a treatment site, and

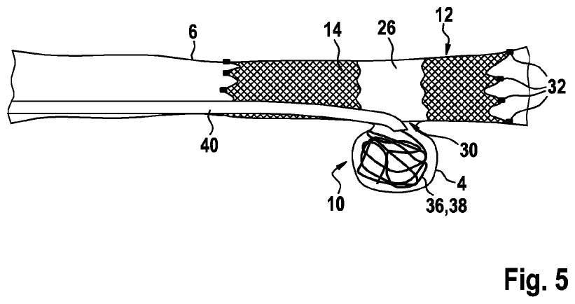

FIG. 5 shows a side view of a preferred embodiment of the medical system in accordance with the invention, in the case of permanent implantation of the covering device at a treatment site.

In the figures, parts with identical actions are always designated by the same reference numerals.

The medical set 2 shown in FIG. 1 is used for the treatment of aneurysms 4 and is shown in FIG. 1 in a state disposed inside a vessel 6.

The medical set 2 has a main catheter 8 as well as a covering device 12 which can be moved through the main catheter 8 to a treatment site 10. The treatment site 10 is preferably a site along the length of the vessel 6 at which the aneurysm 4 is located. The covering device 12 is used for temporary or permanent covering of the aneurysm 4, wherein the covering device 12 comprises a self-expandable mesh structure 14 produced from webs 16 (see FIG. 2). The mesh structure 14 is preferably produced from a shape memory material with a view to it being self-expandable.

Furthermore, the covering device 12 is permanently or mechanically detachably connected to a transport wire 15 which can be displaced inside the main catheter 8. The mesh structure 14 and therefore the covering device 12 when in a non-expanded state can be pushed and withdrawn through the main catheter 8 by means of this transport wire 15.

In the expanded state, the mesh structure 14 is perfusible by blood along the longitudinal axis, i.e. in and against a direction of flow F.

In particular, the mesh structure 14 is disposed inside the vessel 6 in a manner such that the covering 26 is positioned at a level with an opening 30 of the aneurysm 4 so that the latter is covered by the covering 26, wherein at the same time, a flow of blood through the vessel 6 is not stopped. The covering 26 in the exemplary embodiment of FIG. 1 is therefore substantially tubular in configuration, in particular in the manner of a hollow cylinder.

In this regard, the covering 26 is porous in configuration with large pores 28 (see FIG. 3) and perfusible, so that a supply of nutrients is ensured to the cells covered by the covering 26. Alternatively, however, the covering 26 may also be porous and not perfusible by blood.

In this regard, in particular when an embolization means 36 is being positioned (see FIG. 4) inside the aneurysm 4, the covering 26 is used so that after positioning, the embolization means 40 cannot escape from the aneurysm 4 until the blood inside the aneurysm 4 has been clotted because of the embolization means 36 and therefore the aneurysm 4 has been effectively occluded.

Particularly preferably, the covering 26 is configured as an electrospun covering 26.

As can readily be seen in FIG. 1, in addition, the medical set 2 and in particular the mesh structure 14 is provided with radiographic markers 32. The radiographic markers 32 are preferably disposed at cell tips of edge cells 20 (see FIG. 2) of the mesh structure 14. Specifically, the radiographic markers 56 are formed as radiopaque sleeves, for example produced from platinum or gold, which are crimped onto the cell tips of the edge cells 20.

FIG. 2 shows a close-up of a preferred embodiment of the covering device 12 in accordance with the invention. The webs 16 are connected together in one piece, i.e. they are monolithic, and delimit inner cells 18 as well as edge cells 20. Preferably, all of the cells, in particular inner cells 18 and edge cells 20, are respectively delimited by four webs, which together form a diamond shape. These cells which are enclosed on all sides are designated as closed cells. The edge cells 20 at one longitudinal end 22 of the mesh structure 14 form a closed edge cell ring 24 (three edge cells 20 of the edge cell ring 24 are shown encircled) in the circumferential direction which is connected to inner cells 18 on only one side.

Furthermore, the mesh structure 14 is provided with the covering 26 produced from a fabric which has irregularly sized pores 28 (see FIG. 3). At least one inner cell 18, three inner cells 18 in FIG. 1 (in the plane of the figure at the right hand edge of the mesh structure 14) are covering-free.

The construction of the covering 26 can easily be seen in the scanning electron microscope image of FIG. 3. This shows that the covering 26 has a plurality of pores 28 of irregular sizes which are respectively delimited by filaments 34. The electrospinning process forms a plurality of filaments 34 which are orientated in an irregular manner with respect to each other. In this way, the pores 28 are formed. FIG. 3 also shows that the pores 28 have a comparatively small pore size, wherein some pores 28, however, are sufficiently large to ensure perfusion, for example. Specifically, four pores 28 in FIG. 3 have been highlighted; they are more than 30 μm² in size. The density of the pores 28 which are more than 30 μm² in size indicates that the covering has at least 10 pores 28 of this type over an area of 100000 μm².

FIG. 3 also shows that the filaments 34 of the covering 26 cross over each other multiple times. However, a particular feature of the electrospinning process is that in the covering 26, sites are present at which exclusively two, i.e. no more than, two filaments 34 intersect. It is clear from this that the wall thickness of the covering 26 is very small and it is therefore very flexible.

The high flexibility of the covering 26 combination with the high flexibility of the mesh structure 14 ensures that a covering device 12 can be produced which can be introduced into a (blood) vessel 6 through very small delivery catheters. In particular, delivery catheters may be used which have a size of 6 French, in particular at most 5 French, in particular at most 4 French, in particular at most 3 French, in particular at most 2 French. Specifically, the covering device in accordance with the exemplary embodiments described here may be used in catheters which have an internal diameter of at most 1.6 mm, in particular at most 1.0 mm, in particular at most 0.7 mm, in particular at most 0.4 mm.

In particularly preferred variations, the layer thickness of the covering 26 is at most 10 μm, in particular at most 8 μm, in particular at most 6 μm, in particular at most 4 μm. In this regard, at most 4, in particular at most 3, in particular at most 2 filaments 34 intersect. In general, inside the electrospun structure of tine covering 26, points of intersection are provided in which only 2 filaments 34 intersect. Preferably, the mesh structure 10 has a cross sectional diameter of between 2.5 mm and 8 mm, particular between 4.5 mm and 6 mm.

FIG. 4 shows a diagrammatically illustrated exemplary embodiment of a medical system in accordance with the invention wherein the covering device (12) has been temporarily implanted at a treatment site 10.

In this regard, the medical system comprises the aforementioned medical set 2 with the main catheter 8 as well as the covering device 12, which has the mesh structure 14 and the covering 26. The configuration of the covering 26 here corresponds to the aforementioned embodiment in accordance with FIG. 1.

Furthermore, the medical system has an embolization means 36 which, for example, is formed by a plastically deformable wire 38 or by a liquid. The embolization means 36 is placed inside the aneurysm 4 by means of an additional catheter 40 which is also part of the medical system.

In order to place the embolization means 36, in accordance with the illustrated embodiment of the medical system, the additional catheter 40 is disposed substantially parallel to, i.e. alongside the medical set 2 inside the vessel 6. In this regard, the additional catheter 40 and in particular a tip of the additional catheter 40 is “pushed” into the aneurysm 4 between a vessel wall and the covering 26 in order to place the embolization means 36 in the form of the plastically deformable wire 38 (also known as a “coil”) in it. During this and—in the event of permanent implantation—afterwards, the covering 26 prevents the embolization means 36 from escaping from the aneurysm 4, for example because of the flow of blood.

FIG. 5 shows a diagrammatic representation of an exemplary embodiment of a medical system in accordance with the invention in the case of permanent implantation of the covering device (12) at a treatment site 10.

In respect of the structural features, the medical set as well as the covering device 12 substantially correspond to the medical set described above in respect of FIG. 4. In FIG. 5, the covering device 12 is shown already implanted and expanded in the vessel 6. This means that the covering device 12 would already have been brought to the treatment site 10 by means of the transport wire 15 and detached from the transport wire 15 once there.

However, the covering device 12 of FIG. 5 has respective open longitudinal ends and is therefore substantially cylindrical, i.e. tubular in configuration, so as not to influence the flow of blood through the vessel. In the exemplary embodiment of FIG. 5, the covering device 12 therefore acts as a flow diverter.

### LIST OF REFERENCE NUMERALS

2 medical set

4 aneurysm

6 vessel

8 main catheter

10 treatment site

12 covering device

14 mesh structure

15 transport wire

16 web

18 inner cell

20 edge cell

22 longitudinal end

24 edge cell ring

26 covering

28 pore

30 opening of aneurysm

32 radiographic marker

34 filaments

36 embolization means

38 wire

40 additional catheter

F direction of flow

1-15. (canceled)

16. A medical set for treatment of an aneurysm comprising:
a main catheter; and
a covering device adapted to move through the main catheter to a treatment site to cover the aneurysm, the covering device adapted to connect to a transport wire, the covering device including a self-expandable mesh structure having webs connected together in one piece and that delimit inner cells and edge cells, wherein at a longitudinal end of the mesh structure, the edge cells form a closed edge cell ring connected to the inner cells on only one side,
wherein the mesh structure is provided with a covering produced from a fabric having pores of irregular sizes, and wherein at least one inner cell of the mesh structure is at least partially covering-free.

17. The medical set according to claim 16 wherein the covering is configured as an electrospun covering.

18. The medical set according to claim 16 wherein the covering device is reversibly connectable to the transport wire and, in an expanded state, is open on both end sides and perfusible by blood.

19. The medical set according to claim 16 wherein the webs of the inner cells are respectively associated with one of a further inner cell or edge cell, and wherein the edge cells respectively have two webs which are not associated with one of other inner cells or edge cells.

20. The medical set according to claim 16 wherein the covering ends at the webs of the inner cells which are free from the covering such that the covering does not protrude into the at least one inner cell.

21. The medical set according to claim 16 wherein the covering at least partially overlaps the webs of the at least one inner cell such that part of the covering protrudes into the at least one inner cell which is free from the covering.

22. The medical set according to claim 16 wherein a plurality of inner cells immediately adjacent in a circumferential direction of the mesh structure is covering-free.

23. The medical set according to claim 16 wherein a plurality of inner cells immediately adjacent in a longitudinal direction of the mesh structure is covering-free.

24. The medical set according to claim 16 wherein the covering extends by at most 50% over a circumference of the mesh structure.

25. The medical set according to claim 16 wherein the covering has at least 10 pores with a size of at least 15 μm² over an area of 100,000 μm².

26. A method for production of a medical set for treatment of an aneurysm comprising:
providing a main catheter and a covering device adapted to move through the main catheter to a treatment site to cover the aneurysm, the covering device adapted to connect to a transport wire;
providing a self-expandable mesh structure of the covering device, the mesh structure having webs connected together in one piece and that delimit inner cells and edge cells, wherein at a longitudinal end of the mesh structure, the edge cells form a closed edge cell ring connected to the inner cells on only one side;
providing a covering of the mesh structure, the covering produced from a fabric having pores of irregular sizes;
applying the covering to all inner cells using an electrospinning process; and
cutting at least one inner cell free using a cutting tool, wherein the cutting tool is guided along the webs of the inner cells to be cut free so that the at least one inner cell is at least partially covering-free.

27. A medical system for treatment of an aneurysm comprising:
a main catheter; and
a covering device adapted to move through the main catheter to a treatment site to cover the aneurysm, the covering device adapted to connect to a transport wire, the covering device including a self-expandable mesh structure having webs connected together in one piece and that delimit inner cells and edge cells, wherein at a longitudinal end of the mesh structure, the edge cells form a closed edge cell ring connected to the inner cells on only one side and with at least one embolization means for placement in the aneurysm.

28. The medical system according to claim 27 wherein the covering device is detachably connectable to the transport wire for permanent implantation of the covering device at the treatment site and wherein in an expanded state, the covering device has open longitudinal ends.

29. The medical system according to claim 27 wherein the covering device is non-detachably connected to the transport wire for temporary implantation of the covering device at the treatment site.

30. The medical system of claim 27 further comprising:
an additional catheter configured to deliver the embolization means into the aneurysm, wherein the additional catheter is relatively movable with respect to the main catheter, one of independently of or in opposition to the main catheter.

31. The medical system according to claim 27 wherein the mesh structure is provided with a covering produced from a fabric having pores of irregular sizes, and wherein at least one inner cell of the mesh structure is at least partially covering-free.

32. The medical system according to claim 31 wherein the covering is configured as an electrospun covering.

33. The medical system according to claim 31 wherein the covering ends at the webs of the inner cells which are free from the covering such that the covering does not protrude into the at least one inner cell.

34. The medical system according to claim 31 wherein the covering at least partially overlaps the webs of the at least one inner cell such that part of the covering protrudes into the at least one inner cell which is free from the covering.

35. The medical system according to claim 27 wherein the covering device is reversibly connectable to the transport wire and, in an expanded state, is open on both end sides and perfusible by blood.
